(19) United States
(12) Patent Application Publication
(10) Pub. No.: US 2026/0027371 A1
(43) Pub. Date: Jan. 29, 2026

(54) COCHLEAR IMPLANT SYSTEM FOR MONITORING PROPERTIES ASSOCIATED WITH A SKIN FLAP OF A RECIPIENT

(71) Applicant: Advanced Bionics LLC, Valencia, CA (US)

(72) Inventors: Joachim Thiemann, Hannover (DE); Manuel Christoph Kohl, Hannover (DE); Raphael S. Koning, Wedemark (DE)

(21) Appl. No.: 18/787,093

(22) Filed: Jul. 29, 2024

(51) Int. Cl. A61N 1/372; A61N 1/05

(52) U.S. Cl. CPC A61N 1/37223; A61N 1/0541

(57) ABSTRACT

An exemplary cochlear implant system includes a cochlear implant and a processing unit communicatively coupled with the cochlear implant. The cochlear implant may include a first optical element implanted within a recipient and positioned toward an inner surface of a skin flap of the recipient. The processing unit may cause an optical signal transmission between the first optical element and a second optical element included in an external device positioned toward outer surface of the skin flap relative to the first optical element. The optical signal transmission may include a transmission of an optical signal through the skin flap between the inner surface and the outer surface of the skin flap. Based on the optical signal transmission, one or more properties associated with the skin flap of the recipient may be determined.

### BACKGROUND INFORMATION

Cochlear implant systems are used to provide, restore, and/or improve the sensation of hearing for cochlear implant recipients suffering from certain types of hearing loss. To this end, cochlear implant systems typically include: 1) a cochlear implant implanted within the recipient and configured to apply electrical stimulation to cochlear tissue of the recipient, 2) a headpiece that rests on the head over an implantation site of the cochlear implant to allow transcutaneous communication with the cochlear implant, and 3) a sound processor communicatively coupled with the cochlear implant by way of the headpiece and configured to provide power and stimulation parameters to direct the cochlear implant to apply the electrical stimulation to the recipient.

Typically, the headpiece is located at the implantation site of the cochlear implant such that a skin flap of the recipient is positioned between the cochlear implant and the headpiece. In some instances, the headpiece may be held to the cochlear implant by a magnetic force, which may compress the skin flap and, over time, cause the health of the skin flap to deteriorate. Such deterioration of the health of the skin flap may lead to tissue damage associated with the skin flap and/or discomfort for the recipient. In instances where deterioration of the skin flap occurs, the cochlear implant may need to be removed and/or relocated from the skin flap, which may be costly and/or time consuming.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 shows an illustrative configuration of a cochlear implant system.

FIG. 2 shows another illustrative configuration of a cochlear implant system.

FIG. 3 shows another illustrative configuration of a cochlear implant system.

FIG. 4 shows another illustrative configuration of a cochlear implant system.

FIG. 5 shows an illustrative method for monitoring one or more properties associated with a skin flap.

FIG. 6 shows another illustrative configuration of a cochlear implant system.

FIG. 7A shows an illustrative configuration of the cochlear implant system of FIG. 6 positioned about a skin flap.

FIG. 7B shows an illustrative configuration of the cochlear implant system of FIG. 6 performing an optical signal transmission.

FIG. 8 shows another illustrative method for monitoring one or more properties associated with a skin flap.

FIG. 9 shows another illustrative method for monitoring one or more properties associated with a skin flap.

FIG. 10 shows another illustrative method for monitoring one or more properties associated with a skin flap.

FIG. 11 shows another illustrative configuration of a cochlear implant system.

FIG. 12 shows another illustrative configuration of a cochlear implant system.

FIG. 13 shows another illustrative configuration of a cochlear implant system.

FIG. 14 shows an illustrative computing device.

### DETAILED DESCRIPTION

Systems and methods for monitoring one or more properties associated with a skin flap using a cochlear implant system are described herein. For example, a cochlear implant system may include a cochlear implant having a first optical element configured to be implanted within a recipient and positioned toward an inner surface of a skin flap of the recipient. The cochlear implant system may further include a processing unit communicatively coupled with the cochlear implant and configured to cause an optical signal transmission between the first optical element and a second optical element included in an external device and configured to be positioned toward outer surface of the skin flap relative to the first optical element, the optical signal transmission may include a transmission of an optical signal through the skin flap between the inner surface and the outer surface of the skin flap. Based on the optical signal transmission, the processing unit may further be configured to determine one or more properties (e.g., a thickness, a blood oxygenation, a pulse rate, a blood vessel size, a blood flow, a blood perfusion, a blood pressure, blood circulation, light absorption, etc.) associated with the skin flap of the recipient.

An additional illustrative cochlear implant system may comprise a cochlear implant including a first optical element configured to be implanted within a recipient and positioned toward an inner surface of a skin flap of the recipient, a headpiece including a second optical configured to be positioned external of the recipient toward an outer surface of the skin flap relative to the first optical element, and a processing unit communicatively coupled with the cochlear implant and the headpiece. The processing unit may be configured to: cause an optical signal transmission between the first optical element and the second optical element, the optical signal transmission including a transmission of an optical signal through the skin flap between the inner surface and the outer surface of the skin flap, and determine, based on the optical signal transmission, one or more properties associated with the skin flap of the recipient.

An illustrative system may comprise a memory storing instructions and a processor configured to execute the instructions to perform a process. The process may include: causing an optical source included in either an implantable device implanted within a recipient or an external device external to the recipient to transmit an optical signal towards a first surface of skin flap of a recipient, causing an optical sensor included in the other of the implantable device or the external device to attempt to detect the optical signal from a second surface of the skin flap of the recipient and through the skin flap, and determining, based on sensor data output by the optical sensor and representative of one or more optical signals detected by the optical sensor during a time period corresponding to the transmitting of the optical signal by the optical source, one or more properties associated with the skin flap of the recipient.

The systems and methods described herein may provide various benefits to cochlear implant recipients. For example, systems and methods such as those described herein may monitor the health of the skin flap by determining, based on an optical signal transmission, one or more properties associated with the skin flap. Such a determination of the one or more properties associated with the skin flap may assist a clinician in evaluating the health of the skin flap and/or automatically identify changes associated with the one or more properties indicative of a deterioration of the skin flap in a clinically effective manner than minimizes the analytical burden of a clinician. Systems and methods such as those described herein may further provide benefit to the recipient such as by providing a notification associated with a change in the one or more properties of the skin flap and/or decreasing a strength of one or more magnets included in the cochlear implant system, which may prevent deterioration of the skin flap. By preventing deterioration of the skin flap, the cochlear implant system may continue to operate at the skin flap such that removal and/or relocation of the cochlear implant may be avoided.

Various embodiments will now be described in more detail with reference to the figures. The disclosed systems and methods may provide one or more of the benefits mentioned above and/or various additional and/or alternative benefits that will be made apparent herein.

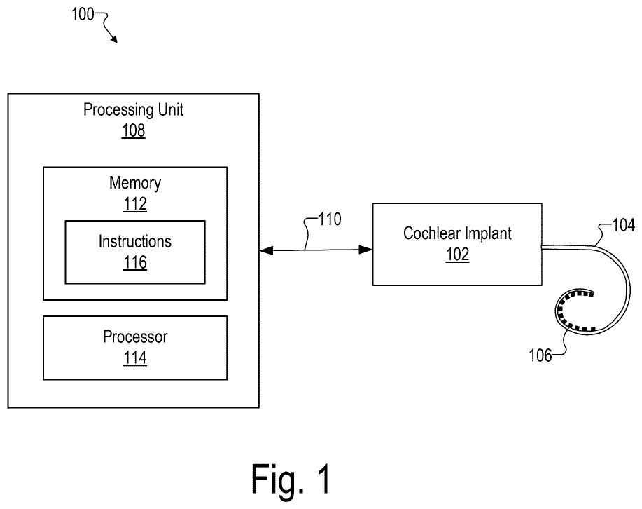

FIG. 1 illustrates an exemplary cochlear implant system 100 configured to be used by a recipient. As shown, cochlear implant system 100 includes a cochlear implant 102, an electrode lead 104 physically coupled to cochlear implant 102 and having an array of electrodes 106, and a processing unit 108 configured to be communicatively coupled to cochlear implant 102 by way of a communication link 110. Cochlear implant system 100 may include additional or alternative components as may serve a particular implementation.

The cochlear implant system 100 shown in FIG. 1 is unilateral (i.e., associated with only one ear of the recipient). Alternatively, a bilateral configuration of cochlear implant system 100 may include separate cochlear implants and electrode leads for each ear of the recipient. In the bilateral configuration, processing unit 108 may be implemented by a single processing unit configured to interface with both cochlear implants or by two separate processing units each configured to interface with a different one of the cochlear implants.

Cochlear implant 102 may be implemented by any suitable type of implantable stimulator. For example, cochlear implant 102 may be implemented by an implantable cochlear stimulator. Additionally or alternatively, cochlear implant 102 may be implemented by a bone conduction implant, a middle ear implant, a brainstem implant (e.g., an auditory brainstem implant) and/or any other type of device that may be implanted within the recipient and configured to apply electrical stimulation to one or more stimulation sites located along an auditory pathway of the recipient.

In some examples, cochlear implant 102 is configured to generate electrical stimulation representative of an audio signal processed by processing unit 108 in accordance with one or more stimulation parameters transmitted to cochlear implant 102 by processing unit 108. Cochlear implant 102 may be further configured to apply the electrical stimulation to one or more stimulation sites (e.g., one or more intracochlear locations) within the recipient by way of one or more electrodes 106 on electrode lead 104. In some examples, cochlear implant 102 may include a plurality of independent current sources each associated with a channel defined by one or more of electrodes 106. In this manner, different stimulation current levels may be applied to multiple stimulation sites simultaneously by way of multiple electrodes 106.

Cochlear implant 102 may additionally or alternatively be configured to generate, store, and/or transmit data. For example, cochlear implant may use one or more electrodes 106 to record one or more signals (e.g., one or more voltages, impedances, evoked responses within the recipient, and/or other measurements) and transmit, by way of communication link 110, data representative of the one or more signals to processing unit 108. In some examples, this data is referred to as back telemetry data.

Electrode lead 104 may be implemented in any suitable manner. For example, a distal portion of electrode lead 104 may be pre-curved such that electrode lead 104 conforms with the helical shape of the cochlea after being implanted. Electrode lead 104 may alternatively be naturally straight or of any other suitable configuration.

In some examples, electrode lead 104 includes a plurality of wires (e.g., within an outer sheath) that conductively couple electrodes 106 to one or more current sources within cochlear implant 102. For example, if there are n electrodes 106 on electrode lead 104 and n current sources within cochlear implant 102, there may be n separate wires within electrode lead 104 that are configured to conductively connect each electrode 106 to a different one of the n current sources. Exemplary values for n are 8, 12, 16, or any other suitable number.

Electrodes 106 are located on at least a distal portion of electrode lead 104. In this configuration, after the distal portion of electrode lead 104 is inserted into the cochlea, electrical stimulation may be applied by way of one or more of electrodes 106 to one or more intracochlear locations. One or more other electrodes (e.g., including a ground electrode, not explicitly shown) may also be disposed on other parts of electrode lead 104 (e.g., on a proximal portion of electrode lead 104) to, for example, provide a current return path for stimulation current applied by electrodes 106 and to remain external to the cochlea after the distal portion of electrode lead 104 is inserted into the cochlea. Additionally or alternatively, a housing of cochlear implant 102 may serve as a ground electrode for stimulation current applied by electrodes 106. In certain examples, electrode lead 104 may alternatively be referred to as an electrode array.

Processing unit 108 may be configured to interface with (e.g., control and/or receive data from) cochlear implant 102. For example, processing unit 108 may transmit commands (e.g., stimulation parameters and/or other types of operating parameters in the form of data words included in a forward telemetry sequence) to cochlear implant 102 by way of communication link 110. Processing unit 108 may additionally or alternatively provide operating power to cochlear implant 102 by transmitting one or more power signals to cochlear implant 102 by way of communication link 110. Processing unit 108 may additionally or alternatively receive data from cochlear implant 102 by way of communication link 110. Communication link 110 may be implemented by any suitable number of wired and/or wireless bidirectional and/or unidirectional links.

As shown, processing unit 108 includes a memory 112 and a processor 114 configured to be selectively and communicatively coupled to one another. In some examples, memory 112 and processor 114 may be distributed between multiple devices and/or multiple locations as may serve a particular implementation.

Memory 112 may be implemented by any suitable non-transitory computer-readable medium and/or non-transitory processor-readable medium, such as any combination of non-volatile storage media and/or volatile storage media. Exemplary non-volatile storage media include, but are not limited to, read-only memory, flash memory, a solid-state drive, a magnetic storage device (e.g., a hard drive), ferroelectric random-access memory (“RAM”), and an optical disc. Exemplary volatile storage media include, but are not limited to, RAM (e.g., dynamic RAM).

Memory 112 may maintain (e.g., store) executable data used by processor 114 to perform one or more of the operations described herein. For example, memory 112 may store instructions 116 that may be executed by processor 114 to perform any of the operations described herein. Instructions 116 may be implemented by any suitable application, program (e.g., sound processing program), software, code, and/or other executable data instance. Memory 112 may also maintain any data received, generated, managed, used, and/or transmitted by processor 114.

Processor 114 may be configured to perform (e.g., execute instructions 116 stored in memory 112 to perform) various operations with respect to cochlear implant 102. To illustrate, processor 114 may be configured to control an operation of cochlear implant 102. For example, processor 114 may receive an audio signal (e.g., by way of a microphone communicatively coupled to processing unit 108, a wireless interface (e.g., a Bluetooth interface), and/or a wired interface (e.g., an auxiliary input port)). Processor 114 may process the audio signal in accordance with a sound processing program (e.g., a sound processing program stored in memory 112) to generate appropriate stimulation parameters. Processor 114 may then transmit the stimulation parameters to cochlear implant 102 to direct cochlear implant 102 to apply electrical stimulation representative of the audio signal to the recipient.

In some implementations, processor 114 may also be configured to apply acoustic stimulation to the recipient. For example, a receiver (also referred to as a loudspeaker) may be optionally coupled to processing unit 108. In this configuration, processor 114 may deliver acoustic stimulation to the recipient by way of the receiver. The acoustic stimulation may be representative of an audio signal (e.g., an amplified version of the audio signal), configured to elicit an evoked response within the recipient, and/or otherwise configured. In configurations in which processor 114 is configured to both deliver acoustic stimulation to the recipient and direct cochlear implant 102 to apply electrical stimulation to the recipient, cochlear implant system 100 may be referred to as a bimodal hearing system and/or any other suitable term.

Processor 114 may be additionally or alternatively configured to receive and process data generated by cochlear implant 102. For example, processor 114 may receive data representative of a signal recorded by cochlear implant 102 using one or more electrodes 106 and, based on the data, adjust one or more operating parameters of processing unit 108. Additionally or alternatively, processor 114 may use the data to perform one or more diagnostic operations with respect to cochlear implant 102 and/or the recipient.

Other operations may be performed by processor 114 as may serve a particular implementation. In the description provided herein, any references to operations performed by processing unit 108 and/or any implementation thereof may be understood to be performed by processor 114 based on instructions 116 stored in memory 112.

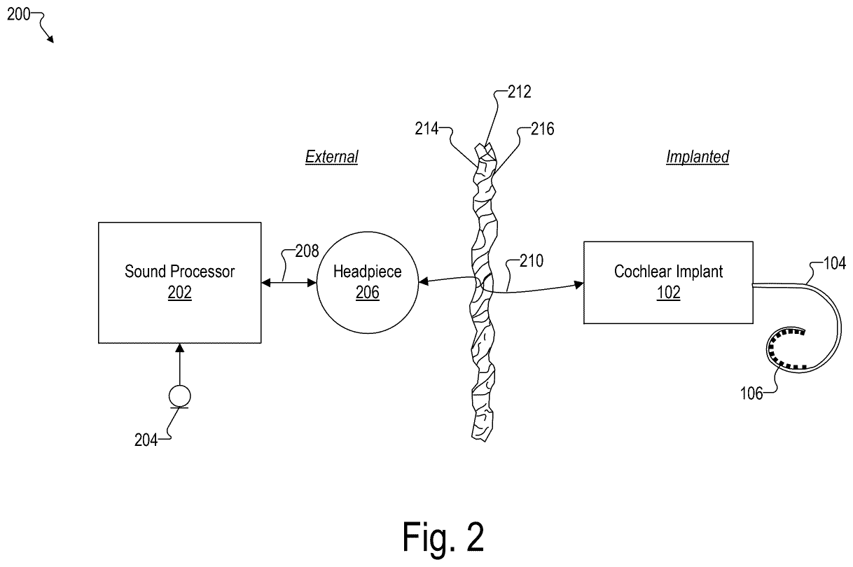

Processing unit 108 may be implemented by one or more devices configured to interface with cochlear implant 102. To illustrate, FIG. 2 shows an exemplary configuration 200 of cochlear implant system 100 in which processing unit 108 is implemented by a sound processor 202 configured to be located external to the recipient. Configuration 200 may include additional or alternative components as may serve a particular implementation. In configuration 200, sound processor 202 is communicatively coupled to a microphone 204 and to a headpiece 206 that are both configured to be located external to the recipient. In configuration 200, cochlear implant system 100 may further include various components configured to be implanted within the recipient including cochlear implant 102 and electrode lead 104.

Sound processor 202 may be implemented by any suitable device that may be worn or carried by the recipient. For example, sound processor 202 may be implemented by a behind-the-ear (“BTE”) unit configured to be worn behind and/or on top of an ear of the recipient. Additionally or alternatively, sound processor 202 may be implemented by an off-the-ear unit (also referred to as a body worn device) configured to be worn or carried by the recipient away from the ear. Additionally or alternatively, at least a portion of sound processor 202 is implemented by circuitry within headpiece 206.

Microphone 204 is configured to detect one or more audio signals (e.g., that include speech and/or any other type of sound) in an environment of the recipient. Microphone 204 may be implemented in any suitable manner. For example, microphone 204 may be implemented by a microphone that is configured to be placed within the concha of the ear near the entrance to the ear canal, such as a T-MIC™ microphone from Advanced Bionics. Such a microphone may be held within the concha of the ear near the entrance of the ear canal during normal operation by a boom or stalk that is attached to an ear hook configured to be selectively attached to sound processor 202. Additionally or alternatively, microphone 204 may be implemented by one or more microphones in or on headpiece 206, one or more microphones in or on a housing of sound processor 202, one or more beam-forming microphones, and/or any other suitable microphone as may serve a particular implementation.

Headpiece 206 may be selectively and communicatively coupled to sound processor 202 by way of a communication link 208 (e.g., a cable or any other suitable wired or wireless communication link), which may be implemented in any suitable manner. Headpiece 206 may include an external antenna (e.g., a coil and/or one or more wireless communication components) configured to facilitate selective wireless coupling of sound processor 202 to cochlear implant 102. Headpiece 206 may additionally or alternatively be used to selectively and wirelessly couple any other external device to cochlear implant 102. To this end, headpiece 206 may be configured to be affixed to the recipient's head and positioned such that the external antenna housed within headpiece 206 is communicatively coupled to a corresponding implantable antenna (which may also be implemented by a coil and/or one or more wireless communication components) included within or otherwise connected to cochlear implant 102. In this manner, stimulation parameters and/or power signals may be wirelessly and transcutaneously transmitted between sound processor 202 and cochlear implant 102 by way of a wireless communication link 210.

As shown, headpiece 206 is positioned external of a skin flap 212 of a recipient at an outer surface 214 of skin flap 212 and cochlear implant 102 is positioned internal of skin flap 212 at an inner surface 216 of skin flap 212. As used herein, “skin flap” may refer to an area of skin of the recipient, such as an area of skin at an implantation site of cochlear implant system 100 positioned between headpiece 206 and cochlear implant 102. Skin flap 212 may include one or more layers of skin tissue, such as an epidermis, a dermis, and/or a hypodermis. In some instances, the one or more layers of tissue included in skin flap 212 may contain connective tissue, hair follicles, blood vessels, blood, lymphatic vessels, sweat glands, etc.

In configuration 200, sound processor 202 may receive an audio signal detected by microphone 204 by receiving a signal (e.g., an electrical signal) representative of the audio signal from microphone 204. Sound processor 202 may additionally or alternatively receive the audio signal by way of any other suitable interface as described herein. Sound processor 202 may process the audio signal in any of the ways described herein and transmit, by way of headpiece 206, stimulation parameters to cochlear implant 102 to direct cochlear implant 102 to apply electrical stimulation representative of the audio signal to the recipient.

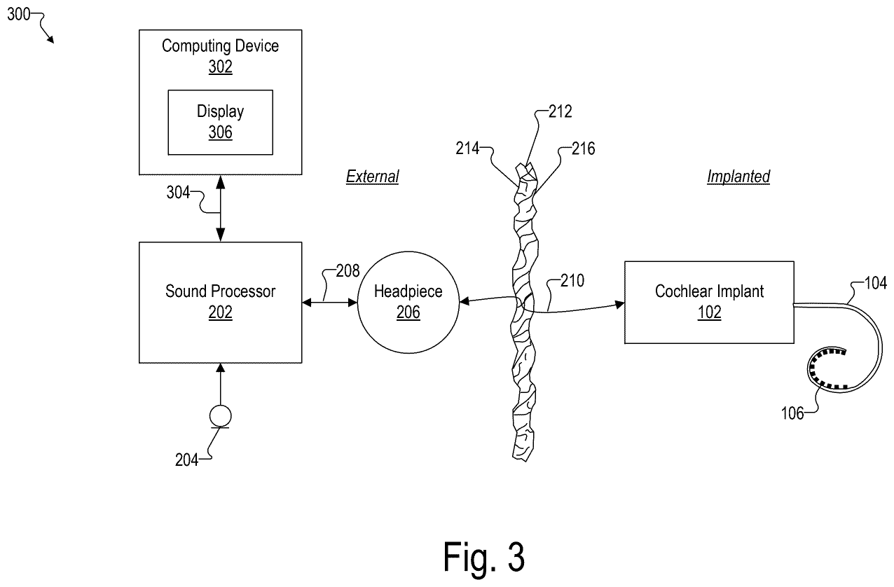

FIG. 3 shows another illustrative configuration 300 of cochlear implant system 100 in which processing unit 108 is implemented by a combination of sound processor 202 and a computing device 302 configured to communicatively couple to sound processor 202 by way of a communication link 304, which may be implemented by any suitable wired or wireless communication link. Configuration 300 may include additional or alternative components as may serve a particular implementation.

Computing device 302 may be implemented by any suitable combination of hardware and software. To illustrate, computing device 302 may be implemented by a mobile device (e.g., a mobile phone, a laptop, a tablet computer, etc.), a desktop computer, and/or any other suitable computing device as may serve a particular implementation. As an example, computing device 302 may be implemented by a mobile device configured to execute an application (e.g., a “mobile app”) that may be used by a user (e.g., the recipient, a clinician, and/or any other user) to control one or more settings of sound processor 202 and/or cochlear implant 102 and/or perform one or more operations (e.g., diagnostic operations) with respect to data generated by sound processor 202 and/or cochlear implant 102.

In some examples, computing device 302 may be configured to control an operation of cochlear implant 102 by transmitting one or more commands to cochlear implant 102 by way of sound processor 202. Likewise, computing device 302 may be configured to receive data generated by cochlear implant 102 by way of sound processor 202. Alternatively, computing device 302 may interface with (e.g., control and/or receive data from) cochlear implant 102 directly by way of a wireless communication link between computing device 302 and cochlear implant 102. In some implementations in which computing device 302 interfaces directly with cochlear implant 102, sound processor 202 may or may not be included in the cochlear implant system.

Computing device 302 is shown as having an integrated display 306. Display 306 may be implemented by a display screen, for example, and may be configured to display content generated by computing device 302. Additionally or alternatively, computing device 302 may be communicatively coupled to an external display device (not shown) configured to display the content generated by computing device 302.

In some examples, computing device 302 represents a fitting device configured to be selectively used (e.g., by a clinician) to fit sound processor 202 and/or cochlear implant 102 to the recipient. In these examples, computing device 302 may be configured to execute a fitting program configured to set one or more operating parameters of sound processor 202 and/or cochlear implant 102 to values that are optimized for the recipient. As such, in these examples, computing device 302 may not be considered to be part of the cochlear implant system. Instead, computing device 302 may be considered to be separate from the cochlear implant system such that computing device 302 may be selectively coupled to the cochlear implant system when it is desired to fit sound processor 202 and/or cochlear implant 102 to the recipient.

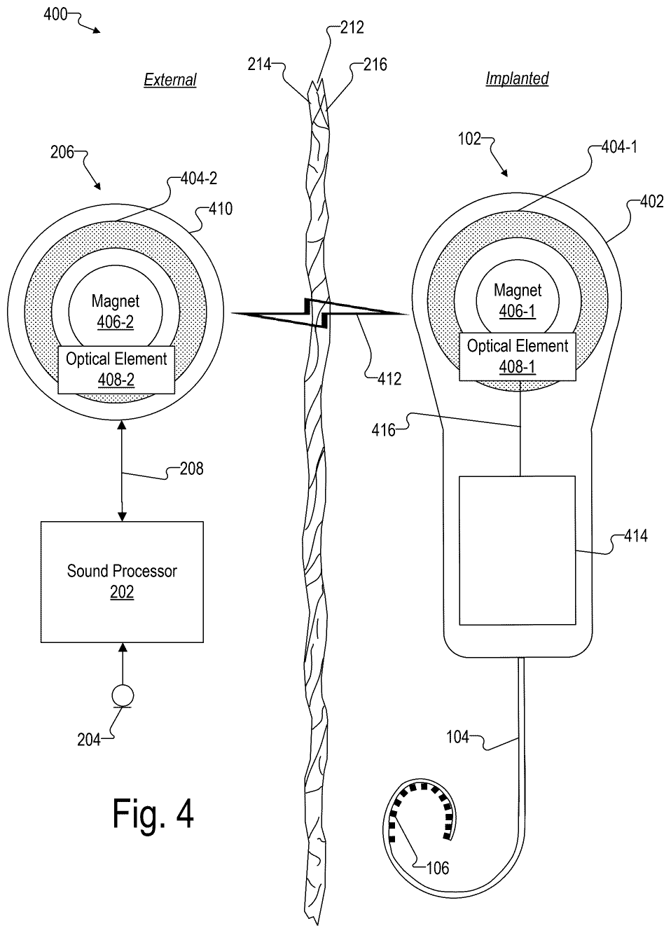

FIG. 4 shows another illustrative configuration 400 of cochlear implant system 100 configured to monitor one or more properties associated with skin flap 212 based on an optical signal transmission. As depicted by the implementation in FIG. 4, cochlear implant 102 includes a housing 402 constructed of a suitable material for enclosing components such as a first antenna 404-1, a first magnet 406-1, and/or one or more first optical elements 408-1. Likewise, headpiece 206 includes a housing 410 constructed of a suitable material for enclosing components such as a second antenna 404-2, a second magnet 406-2, and/or one or more second optical elements 408-2. Configuration 400 may include additional or alternative components as may serve a particular implementation.

First antenna 404-1 included in cochlear implant 102 is configured to be implanted within the recipient and second antenna 404-1 included in headpiece 206 is configured to be located external to the recipient. As shown, antennas 404 may be implemented as coils (and/or one or more other wireless communication components) that are wirelessly and communicatively coupled with each other such that antennas 404 are configured to facilitate selective wireless coupling of sound processor 202 to cochlear implant 102. For example, antennas 404 may be configured to transcutaneously transmit and/or receive signals (e.g., electronic and/or audio signals) between sound processor 202 and cochlear implant 102. As an illustrative example, second antenna 404-2 included in headpiece 206 may be configured to transcutaneously transmit stimulation parameters from sound processor 202 to first antenna 404-1 of cochlear implant 102. Such outgoing transmission to cochlear implant 102 may be referred to as forward telemetry. Additionally, in some examples, first antenna 404-1 may further facilitate backward telemetry in which sound processor 202 receives a transcutaneous transmission sent by first antenna 404-1 of cochlear implant 102 to second antenna 404-2 in a similar way. In these examples, the signal may be provided to and used by sound processor 202 such as to perform sound processing operations and/or monitor one or more properties associated with skin flap 212.

One or more couplings may be used to hold headpiece 206 to the head of the recipient at the implantation site of cochlear implant 102. As shown, the couplings may be implemented by one or more magnets 406 (e.g., magnets 406-1 to 406-2) configured to be selectively coupled such as by a magnetic force 412. For example, cochlear implant 102 may be implanted such that first magnet 406-1 included in cochlear implant 102 is positioned at inner surface 216 of skin flap 212. Headpiece 206 may include a second magnet 406-2 configured to be positioned at outer surface 214 of skin flap 212 relative to first magnet 406-1 of cochlear implant 102. Accordingly, headpiece 206 may be worn on the head of the recipient at a location that is off the ear (i.e., that is not directly behind the ear and that does not touch the ear or rely on the ear to be held in place). Headpiece 206 may be held at outer surface 214 of skin flap 212 by magnetic force 412 acting transcutaneously through skin flap 212 between first magnet 406-1 included in cochlear implant 102 and second magnet 406-2 included in headpiece 206.

In some instances, magnets 406 are selectively couplable such that headpiece 206 may be selectively coupled with cochlear implant 102. This may allow the recipient to remove and/or reposition headpiece 206 relative to cochlear implant 102 as desired. For example, to remove headpiece 206, the recipient may grasp headpiece 206 and pull headpiece 206 away from the implantation site of cochlear implant 102 (e.g., to decrease magnetic force 412 as headpiece 206 is pulled away and decouple magnets 406). The recipient may then replace headpiece 206 relative to cochlear implant 102 by repositioning headpiece 206 toward the implantation site (e.g., to increase magnetic force 412 as headpiece 206 is positioned toward the implantation site and couple magnets 406). In some implementations, magnetic force 412 may be adjustable such as by increasing and/or decreasing a magnetic strength of first magnet 406-1 and/or second magnet 406-2.

While magnets 406 provide one optional way for headpiece 206 to be held in place on the head, other implementations of headpiece 206 may employ other methods of being held in place such as headbands, magnets separate from headpiece 206 and/or cochlear implant 102, etc. As such, magnets 406 may be an optional component and housing 402 of headpiece 206 may be configured to be worn on the head at the location off the ear in any manner as may serve a particular implementation.

One or more optical elements 408 (e.g., optical elements 408-1 to 408-2) may be used to perform an optical signal transmission transcutaneously through skin flap 212 between the one or more optical elements 408. For example, cochlear implant 102 includes a first optical element 408-1 positioned toward inner surface 216 of skin flap 212 and headpiece 206 includes a second optical element 408-2 positioned toward outer surface 214 of skin flap 212 relative to first optical element 408-1. The optical signal transmission may include a transmission of one or more optical signals (e.g., electromagnetic (EM) energy waves, such as light, infrared, visible light, ultraviolet, radio waves, X-rays, gamma rays, microwaves, etc.) from first optical element 408-1 and/or second optical element 408-2 and through skin flap 212 between inner surface 216 and outer surface 214. The one or more optical signals may be detected by first optical element 408-1 and/or second optical element 408-2.

First optical element 408-1 and/or second optical element 408-2 may include one or more optical emitters configured to transmit the one or more optical signals. In some implementations, the one or more optical emitters may be implemented by one or more light sources (e.g., a light-emitting diodes (LEDs), infrared transmitters, etc.) configured to emit light, such as light in a visible range (e.g., light having a wavelength from about 380 nanometers to about 750 nanometers), light in an infrared (IR) range (e.g., light having a wavelength from about 700 nanometers to about 1 millimeter), and/or light including any other suitable wavelengths absorbable by skin flap 212. The one or more light sources may be configured to emit light at substantially the same wavelengths and/or different wavelengths. As an illustrative example, the one or more light sources may include one or more multispectral LEDs, each multispectral LED configured to emit light having a plurality of different wavelengths.

First optical element 408-1 and/or second optical element 408-2 may be configured to continuously transmit the one or more optical signals during the optical signal transmission. Additionally or alternatively, first optical element 408-1 and/or second optical element 408-2 may be configured to modulate (e.g., pulse, step, etc.) the one or more optical signals during the optical signal transmission. Such a modulation of the one or more optical signals may facilitate in distinguishing the one or more optical signals from light associated with an environment of cochlear implant system 100, such as ambient light.

First optical element 408-1 and/or second optical element 408-2 may additionally or alternatively include one or more optical sensors configured to attempt to detect the one or more optical signals transmitted by the one or more optical emitters during the optical signal transmission. In instances where the one or more optical emitters includes one or more light sources, the one or more optical sensors may be implemented by one or more light detectors (e.g., photodetectors, charged-coupled devices (CCDs), cameras, etc.) configured to detect light, such as light in a visible range (e.g., light having a wavelength from about 380 nanometers to about 750 nanometers), light in an infrared (IR) range (e.g., light having a wavelength from about 700 nanometers to about 1 millimeter), and/or light including any other suitable wavelengths absorbable by skin flap 212. In some instances, headpiece 206 may be positioned over hair at outer surface 214 of skin flap 212 such that the one or more optical sensors may be configured to detect the one or more optical signals through the hair. The one or more optical sensors may be unfocused and/or include a focusing device (e.g., a lens) configured to focus the optical signal toward the one or more optical sensors.

Cochlear implant system 100 may include any additional or alternative components that may serve a particular implementation. For example, as shown in FIG. 4, cochlear implant 102 may further include electronic circuitry 414 that may be communicatively coupled to one or more of first antenna 404-1, first magnet 406-1, and/or first optical element 408-1 such as for performing the functionality of cochlear implant 102 described above (e.g., receiving one or more stimulation parameters transmitted to cochlear implant 102 by sound processor 202, applying electrical stimulation to one or more stimulation sites by way of one or more electrodes 106, transmitting and/or detecting one or more optical signals, adjusting a magnetic strength associated with first magnet 406-1, etc.). Electronic circuitry 414 may include one or more computing components (e.g., a processor, a memory within which instructions to be performed by the processor may be stored, etc.) and/or other electronic components configured to perform or direct any of the operations described in relation to cochlear implant 102, first antenna 404-1, first magnet 406-1, and/or first optical element 408-1. Electronic circuitry 414 may be communicatively coupled to one or more of first antenna 404-1, first magnet 406-1, and/or first optical element 408-1 by way of a communication link 416 (e.g., a cable or any other suitable wired or wireless communication link), which may be implemented in any suitable manner. In some implementations, communication link 416 may include a printed circuit board (PCB), which may allow first optical element 408-1 to be positioned on the PCB.

Moreover, while FIG. 4 shows sound processor 202 and microphone 204 separate from headpiece 206, sound processor 202 and/or microphone 204 may alternatively be implemented by headpiece 206. For example, headpiece 206 may include electronic circuitry (not shown) such as for performing the functionality of headpiece 206 and/or sound processor 202 described above. (e.g., transmitting one or more stimulation parameters transmitted to cochlear implant 102, transmitting and/or detecting one or more optical signals, adjusting a magnetic strength associated with second magnet 406-2, etc.). The electronic circuitry may include one or more computing components (e.g., a processor, a memory within which instructions to be performed by the processor may be stored, etc.) and/or other electronic components configured to perform or direct any of the operations described in relation to headpiece 206, sound processor 202, microphone 204, second antenna 404-2, second magnet 406-2, and/or second optical element 408-2.

To illustrate, the electronic circuitry may be configured to receive an audio signal (i.e., an analog or digital signal captured by microphone 204), divide the audio signal into a plurality of channels each associated with audio components within different frequency bands, and generate stimulation parameters corresponding with each of the channels. The electronic circuitry may then send the stimulation parameters, along with electrical power configured to power cochlear implant 102, through the recipient's skin to be received by cochlear implant 102 by way of antennas 404. In certain examples, the electronic circuitry may further perform other operations as may serve a particular implementation. For instance, in certain implementations, the electronic circuitry may perform optical signal processing operations that will be described in more detail below.

Additionally or alternatively, microphone 204 described above may be implemented by one or more built-in microphones (not shown) included in headpiece 206. Such built-in microphones may include one or more microphones configured to capture sound presented to the recipient. In particular, the one or more microphones may capture sound as the sound waves propagate to the off-the-ear location at which headpiece 206 is worn. In some examples, audio signals captured by the one or more microphones may be combined in accordance with a beamforming technique to create a directional audio signal. This may be useful, for example, in facilitating the recipient to understand speech originating from a source in one direction (e.g., directly in front of the recipient) while noise is originating from other directions (e.g., from other speakers in a crowded room in which the recipient is located). In certain implementations, housing 410 of headpiece 206 may include one or more openings to allow sound to be captured more directly by the one or more microphones.

Headpiece 206 may include an audio interface (not shown) in addition to or instead of the one or more microphones. The audio interface may be configured to receive an audio signal (e.g., a microphone signal, a line-in signal, etc.) from a source external to headpiece 206. To this end, the audio interface may include any electrical or mechanical components as may serve a particular implementation, and may function in any suitable way. For example, the audio interface may include or be associated with a cable physically connected to an external audio source (e.g., a microphone assembly), or may implement a wireless interface (e.g., a Bluetooth interface or the like) configured to receive audio signals wirelessly. In some examples, the audio interface may include a connector allowing a cable to be removably coupled to headpiece 206. Any audio signal received by way of the audio interface may be provided to the electronic circuitry for processing.

In some instances, skin flap 212 may be compressed between headpiece 206 and cochlear implant 102, such as due to magnetic force 412 holding headpiece 206 to cochlear implant 102. Over time, such compression may cause deterioration of the health of skin flap 212, such as tissue damage and/or loss of blood circulation. To illustrate, a thickness of skin flap 212 between outer surface 214 and inner surface 216 may decrease over time. Accordingly, cochlear implant system 100 may be configured to monitor, based on the optical signal transmission, one or more properties associated with skin flap 212 that may be indicative of the health of skin flap 212.

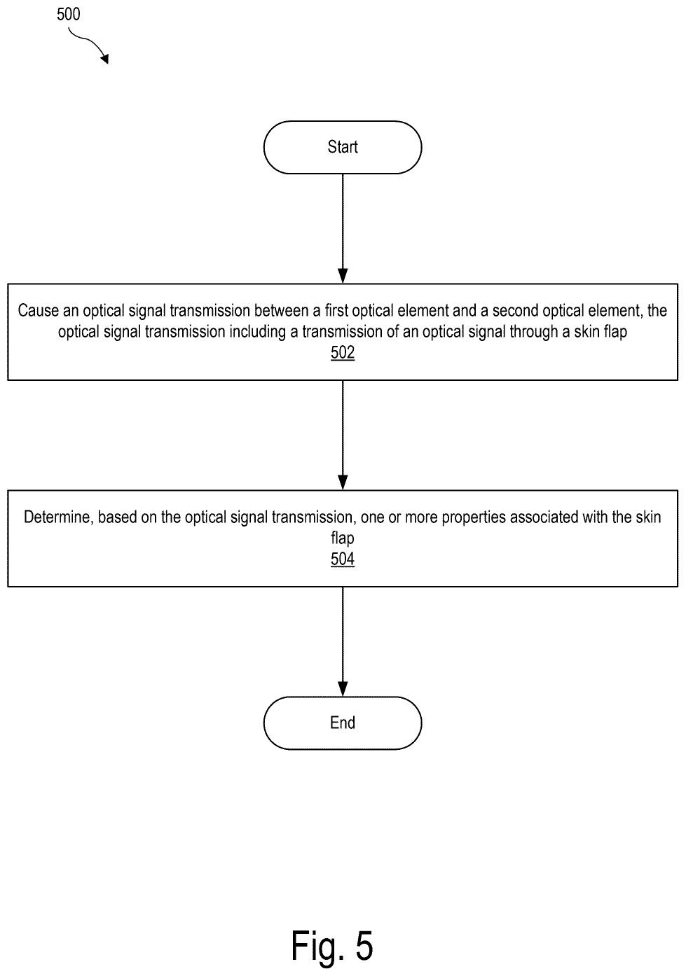

For example, FIG. 5 depicts an illustrative method 500 for monitoring one or more properties associated with a skin flap (e.g., skin flap 212) based on an optical signal transmission of cochlear implant system 100. While FIG. 5 illustrates exemplary operations according to one embodiment, other embodiments may omit, add to, reorder, and/or modify any of the operations shown in FIG. 5. Moreover, each of the operations depicted in FIG. 5 may be performed in any of the ways described herein. One or more of the operations depicted in FIG. 5 may be performed by a processing unit (e.g., processing unit 108) of the cochlear implant system.

As shown, method 500 may include, at operation 502, causing an optical signal transmission between a first optical element (e.g., first optical element 408-1) and a second optical element (e.g., second optical element 408-2). The optical signal transmission may include a transmission of an optical signal through the skin flap between the inner surface and the outer surface of the skin flap. For example, the first optical element and/or the second optical element may include one or more optical emitters configured to transmit the optical signal. In some implementations, the cochlear implant system may cause the one or more optical emitters to emit the optical signal at one or more select wavelengths and/or intensities. The first optical element and/or the second optical element may additionally or alternatively include one or more optical sensors configured to attempt to detect the optical signal during the optical signal transmission. The one or more optical sensors may further be configured to output sensor data representative of the optical signal detected by the one or more optical sensors. In some implementations, the cochlear implant system may cause the optical signal transmission to occur at select intervals (e.g., about every hour, about every 8 hours, about every 12 hours, daily, weekly, monthly, etc.).

The first optical element may be included within a cochlear implant (e.g., cochlear implant 102) configured to be implanted within a recipient and positioned toward an inner surface (e.g., inner surface 216) of the skin flap of the recipient. The second optical element may be included in an external device and configured to be positioned toward an outer surface (e.g., outer surface 214) of the skin flap relative to the first optical element. In some implementations, the external device may be included in the cochlear implant system, such as a headpiece (e.g., headpiece 206) of the cochlear implant system. Additionally or alternatively, the external device may be separate from the cochlear implant system, such as an user end device, a specific headpiece configured to be used by a clinician (e.g., for performing the optical signal transmission and/or fitting of the cochlear implant system), and/or any other suitable device including an optical element. For example, the external device may be selectively held (e.g., by the recipient and/or clinician) at the implantation site of the cochlear implant relative to the first optical element such that the optical signal transmission may be performed between the first optical element and the second optical element.

Method 500 may further include, at operation 504, determining, based on the optical signal transmission, one or more properties associated with the skin flap of the recipient. For example, the one or more properties may be determined based on the sensor data output by the one or more optical sensors. The one or more properties associated with the skin flap may include, but is not limited to, one or more of a thickness, a blood vessel size, or a blood property (e.g., a blood oxygenation, a pulse rate, a blood flow, a blood perfusion, a blood pressure, etc.) associated with the skin flap. Such one or more properties associated with the skin flap may be indicative of the health of the skin flap, such as tissue damage associated with the skin flap and/or blood circulation associated with the skin flap. To illustrate, a greater thickness, blood vessel size, and/or blood property associated with the skin flap may be indicative of a healthy skin flap, while a lower thickness, blood vessel size, and/or blood property may be indicative of an unhealthy skin flap (e.g., the skin flap may have tissue damage).

In some implementations, the determining the one or more properties may be based on a change of intensity of the optical signal between the inner surface and the outer surface of the skin flap during the optical signal transmission. For example, an intensity of the optical signal may decrease as the optical signal is transmitted through tissue of the skin flap between the inner surface and the outer surface (e.g., the intensity of the optical signal at the optical sensor may be less than the initial intensity of the optical signal emitted at the optical emitter). The amount of change in the intensity of the between the inner surface and the outer surface of the skin flap during the optical signal transmission may be indicative of the one or more properties associated with the skin flap. As an illustrative example, a larger decrease in the amount of the intensity of the optical signal may indicate a greater thickness of the skin flap, while a lower decrease in the amount of intensity of the optical signal may indicate a reduced thickness of the skin flap.

In some implementations, an intensity value representative of the change in the intensity of the optical signal during the optical signal transmission may be determined. Such an intensity value may be represented by any suitable metric, such as a discrete value (e.g., a percentage, a level, a range, an amount, etc.). Accordingly, an intensity value associated with a larger change in the intensity may have a higher value within a given range, e.g. between 1 to 100 (e.g., greater than about 50, greater than about 75, and/or greater than about 90) and may indicate a sufficient skin flap thickness, a sufficient amount of blood circulation in the skin flap, and/or a healthy skin flap. Alternatively, an intensity value associated with a lower change may have a lower value (e.g., less than about 49, less than about 25, and/or less than about 10) and may indicate an insufficient skin flap thickness, an insufficient amount of blood circulation in the skin flap, and/or an unhealthy skin flap.

Additionally or alternatively, the determining the one or more properties may be based on an absorption of the optical signal by the skin flap during the optical signal transmission. For example, the optical signal may be absorbed by tissue of the skin flap between the inner surface and the outer surface during the optical signal transmission. The amount of absorption of the optical signal between the inner surface and the outer surface of the skin flap during the optical signal transmission may be indicative of the one or more properties associated with the skin flap. As an illustrative example, a larger amount of absorption of the optical signal may indicate a greater thickness of the skin flap, while a lower amount of absorption of the optical signal may indicate a reduced thickness of the skin flap.

In some implementations, an absorption value representative of the amount of absorption of the optical signal during the optical signal transmission may be determined. Such an absorption value may be represented by any suitable metric, such as a discrete value (e.g., a percentage, a level, a range, an amount, etc.). Accordingly, an absorption value associated with a larger amount of absorption may have a higher value within a given range, e.g. between 1 to 100 (e.g., greater than about 50, greater than about 75, and/or greater than about 90) and may indicate a sufficient skin flap thickness, a sufficient amount of blood circulation in the skin flap, and/or a healthy skin flap. Alternatively, an absorption value associated with a lower amount of absorption may have a lower value (e.g., less than about 49, less than about 25, and/or less than about 10) and may indicate an insufficient skin flap thickness, an insufficient amount of blood circulation in the skin flap, and/or an unhealthy skin flap.

Additionally or alternatively, the determining the one or more properties may be based on a size of one or more blood vessels included within the skin flap. For example, the optical sensor (e.g., a camera) may be configured to detect the size of one or more blood vessels, such as during the optical signal transmission. The size of the one or more blood vessels may be indicative of the one or more properties associated with the skin flap. As an illustrative example, a larger size of blood vessel may indicate a sufficient skin flap thickness, a sufficient amount of blood circulation in the skin flap, and/or a healthy skin flap, while a lower blood vessel size may indicate an insufficient skin flap thickness, an insufficient amount of blood circulation in the skin flap, and/or an unhealthy skin flap. In some implementations, the cochlear implant system may be configured to track the size of the one or more blood vessels such as to determine a change in the size of the one or more blood vessels over time. Additionally or alternatively, the sensor data output by the one or more optical sensors may be provided as a waveform, which may be indicative of one or more blood properties associated with the skin flap. In instances where the optical signal includes a plurality of different wavelengths, a plurality of different properties associated with the skin flap may be determined based on the plurality of different wavelengths.

In some implementations, the determining the one or more properties may include determining a baseline associated with the one or more properties. For example, the cochlear implant system may be calibrated to the recipient based on previous levels of the one or more properties for the recipient. To illustrate, the cochlear implant system may be configured to generate the baseline such as by performing one or more optical signal transmissions over a certain period of time and determining, based on the one or more optical signal transmissions, levels associated with the one or more properties, changes in the intensity of the optical signal, absorption of the optical signal, and/or blood vessel sizes. Additionally or alternatively, the baseline may include reference data based on one or more previous levels associated with the one or more properties, changes in the intensity of the optical signal, absorption of the optical signal, and/or blood vessel sizes of other recipients. In some examples, changes in the intensity of optical signal and/or levels of absorption of the optical signal may correspond to one or more levels of the one or more properties associated with the skin flap. In some examples, the baseline and/or reference data may be stored in memory (e.g., memory 112).

The cochlear implant system may further be configured to track the one or more properties such as by performing additional optical signal transmissions and determining a change in the one or more properties relative to the baseline. In some examples, the change in the one or more properties may be indicative of damage associated with the skin flap. For example, a deviation of the one or more properties from the baseline may indicate a reduced skin flap thickness, a reduced amount of blood circulation in the skin flap, and/or an unhealthy skin flap.

In some implementations, levels associated with the one or more properties and/or a change in the one or more properties relative to the baseline may be compared to a threshold value representative of damage associated with the skin flap. For example, if the levels are below the threshold value, the one or more properties may indicate a sufficient skin flap thickness, a sufficient amount of blood circulation in the skin flap, and/or a healthy skin flap. Alternatively, if the levels meet or exceed the threshold value, the one or more properties may indicate an insufficient skin flap thickness, an insufficient amount of blood circulation in the skin flap, and/or an unhealthy skin flap.

In instances where there is a change in the one or more properties, method 500 may further include providing a notification (e.g., by way of display 306) associated with the one or more properties. For example, the notification may include information associated with the one or more properties, such as a value and/or a level associated with the one or more properties, a change associated with the one or more properties, an indication of the health associated with the skin flap, etc. In some examples, the notification may further include a recommendation, such as decreasing a strength of one or more magnets included in the cochlear implant system and/or removal of one or more components of the cochlear implant system. Additionally or alternatively, the cochlear implant system may be configured to decrease the strength of the one or more magnets, which may reduce the compression of the skin flap between the cochlear implant and the headpiece included in the cochlear implant system and prolong the health of the skin flap. While the illustrated example includes a method for monitoring one or more properties associated with a skin flap (e.g., skin flap 212) based on an optical signal transmission of cochlear implant system 100, the method may be performed for any system including an implantable auditory prosthesis (e.g., a bone conduction implant, a middle ear implant, an auditory brainstem implant, etc.) in addition to or instead of cochlear implant 102, such as an implantable auditory prosthesis including a magnetically coupled external component (e.g., headpiece 206).

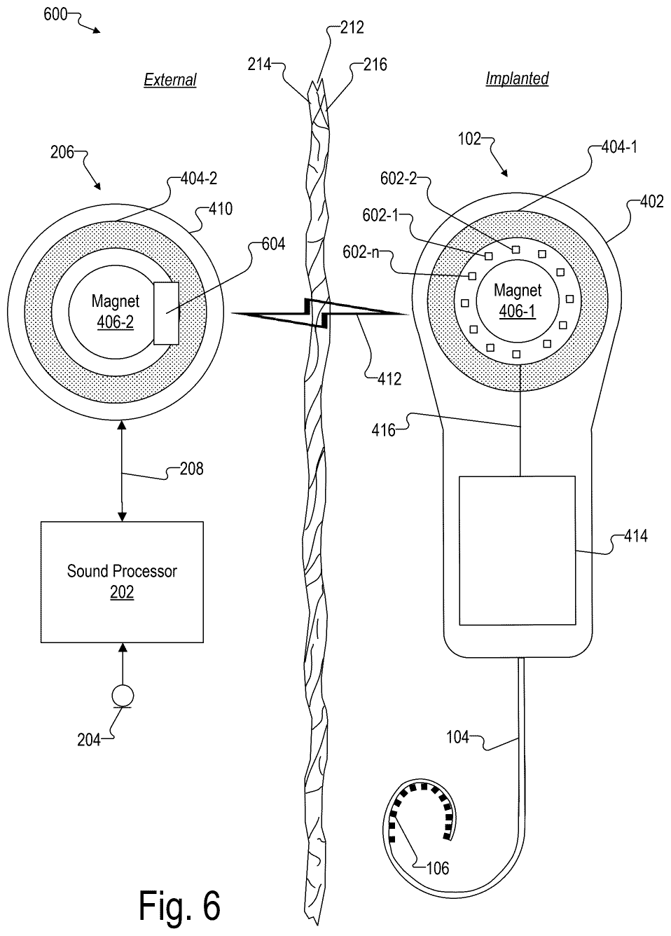

FIG. 6 shows another illustrative configuration 600 of cochlear implant system 100 configured to monitor one or more properties associated with skin flap 212 based on an optical signal transmission. As depicted by the implementation in FIG. 6, first optical element 408-1 is implemented by a one or more optical emitters 602 (e.g., optical emitters 602-1 to 602-n) included in cochlear implant 102 and second optical element 408-2 is implemented by one or more optical sensors 604 included in headpiece 206. Configuration 600 may include additional or alternative components as may serve a particular implementation.

The one or more optical emitters 602 may include one or more light sources, such as one or more LEDs, configured to transmit light. The one or more optical emitters 602 may be positioned at first antenna 404-1 and/or first magnet 406-1. As an illustrative example, a plurality of optical emitters 602 may be positioned in a ring between first antenna 404-1 and first magnet 406-1.

The one or more optical sensors 604 may include one or more light detectors, such as one or more CCD arrays, configured to detect light transmitted by the one or more optical emitters 602. As shown, the one or more optical sensors 604 may be positioned at second antenna 404-2 and/or second magnet 406-2. As an illustrative example, an optical sensor 604 may be positioned in between second antenna 404-2 and second magnet 406-2. While the illustrated example shows one optical sensor 604 included in headpiece 206, in other examples, a plurality of optical sensors 604 may be included in headpiece 206 such as in a ring between second antenna 404-2 and second magnet 406-2.

Accordingly, the one or more optical sensors 604 are positioned to align with the one or more optical emitters 602 when headpiece 206 is coupled with cochlear implant 102 independent of an orientation of headpiece 206 relative to cochlear implant 102. For example, headpiece 206 may be rotated relative to cochlear implant 102 and the one or more optical sensors 604 may still be aligned with the one or more optical emitters 602. This may allow the recipient to remove and/or reposition headpiece 206 as desired. While the illustrated example shows the one or more optical emitters 602 included in cochlear implant 102 and the one or more optical sensors 604 included in headpiece 206, in other examples, the one or more optical emitters 602 may be included in headpiece 206 and the one or more optical sensors 604 may be included in cochlear implant 102.

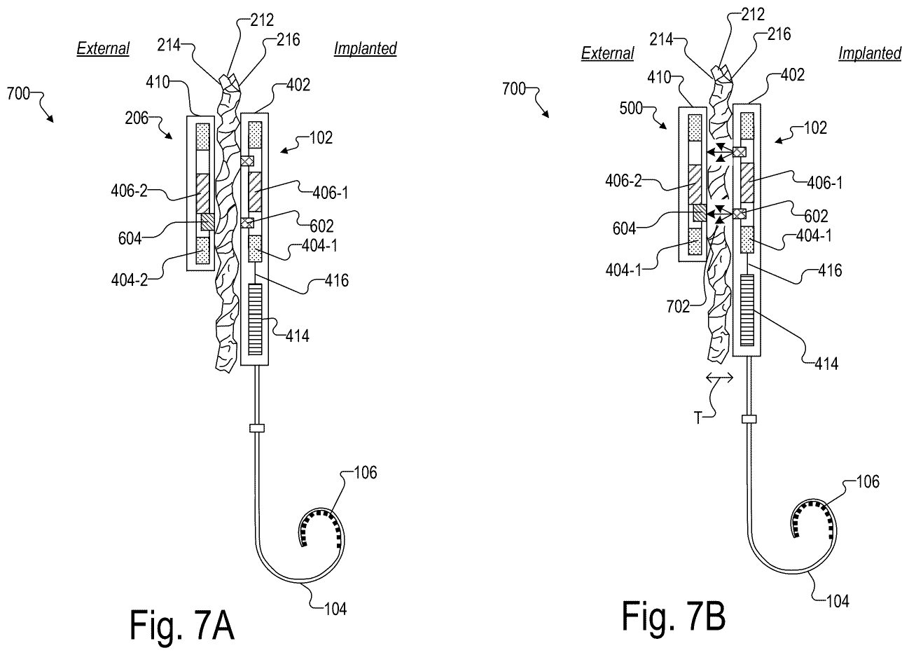

The one or more optical emitters 602 and the one or more optical sensors 604 may be used to perform one or more optical signal transmissions between inner surface 216 and outer surface 214 of skin flap 212 for determining one or more properties associated with skin flap 212. As an illustrative example, FIGS. 7A and 7B show an implementation 700 of cochlear implant system 100 configured to perform an optical signal transmission. As shown in FIG. 7A, cochlear implant 102 is implanted within the recipient to position the one or more optical emitters 602 toward inner surface 216 of skin flap 212. Implementation 700 further includes headpiece 206 positioned external to the recipient and coupled to cochlear implant 102 to position the one or more optical sensors 604 toward outer surface 214 of skin flap 212.

FIG. 7B shows an optical signal transmission between the one or more optical emitters 602 and the one or more optical sensors 604. For example, the one or more optical emitters 602 may be configured to transmit optical signal 702 (e.g., light) at inner surface 216 of skin flap 212 and toward outer surface 214. The one or more optical sensors 604 may be configured to attempt to detect optical signal 702 during the optical signal transmission at outer surface 214. The one or more optical sensors 604 may further be configured to output sensor data representative of optical signal 702 detected by the one or more optical sensors 604 during the optical signal transmission. Based on the sensor data, one or more properties associated with skin flap 212 may be determined. For example, a thickness T of skin flap 212 between inner surface 216 (e.g., at cochlear implant 102) and outer surface 214 (e.g., at headpiece 206) may be determined such as based on a change of intensity of optical signal 702 from inner surface 216 to outer surface 214 and/or an amount of absorption of optical signal 702 from inner surface 216 to outer surface 214.

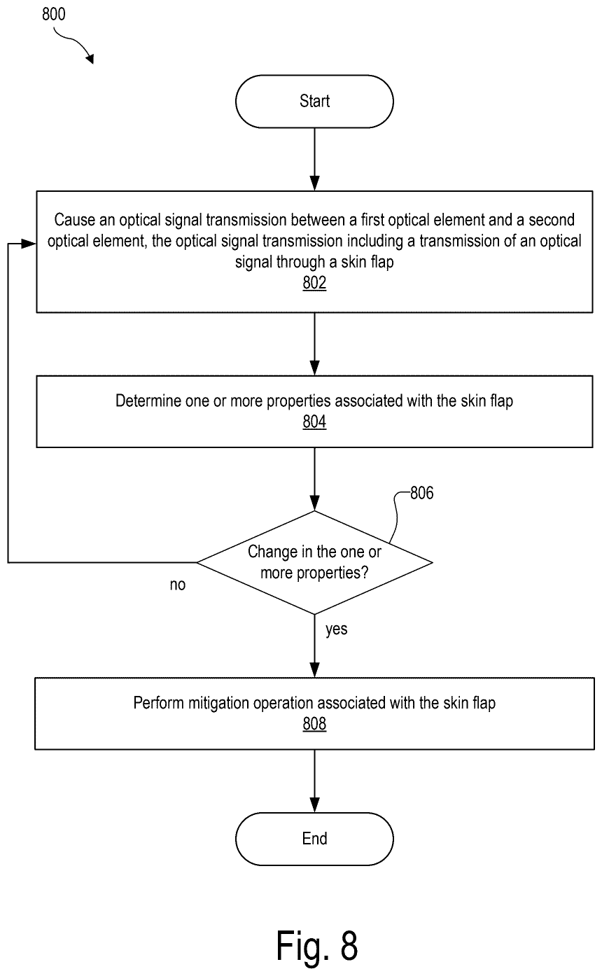

FIG. 8 depicts another illustrative method 800 for monitoring one or more properties associated with a skin flap (e.g., skin flap 212) based on an optical signal transmission of cochlear implant system 100. While FIG. 8 illustrates exemplary operations according to one embodiment, other embodiments may omit, add to, reorder, and/or modify any of the operations shown in FIG. 8. Moreover, each of the operations depicted in FIG. 8 may be performed in any of the ways described herein. One or more of the operations depicted in FIG. 8 may be performed by a processing unit (e.g., processing unit 108) of the cochlear implant system.

As shown, method 800 may include, at operation 802, causing an optical signal transmission between a first optical element (e.g., first optical element 408-1) and a second optical element (e.g., second optical element 408-2). The optical signal transmission may include a transmission of an optical signal through the skin flap between the inner surface and the outer surface of the skin flap. For example, the cochlear implant system may cause one or more optical emitters included in the first and/or second optical elements to emit the optical signal (e.g., at a select wavelength and/or intensity). The cochlear implant system may further cause one or more optical sensors included in the first and/or second optical elements to attempt to detect the optical signal during the optical signal transmission. The one or more optical sensors may further be configured to output sensor data representative of the optical signal detected by the one or more optical sensors.

Method 800 may further include, at operation 804, determining, based on the optical signal transmission, one or more properties associated with the skin flap of the recipient. For example, the one or more properties may be determined based on the sensor data output by the one or more optical sensors. The one or more properties associated with the skin flap may include, but is not limited to, one or more of a thickness, a blood vessel size, or a blood property (e.g., a blood oxygenation, a pulse rate, a blood flow, a blood perfusion, a blood pressure, etc.) associated with the skin flap. Such one or more properties associated with the skin flap may be indicative of the health of the skin flap, such as tissue damage associated with the skin flap and/or blood circulation associated with the skin flap. In some implementations, the determining the one or more properties may be based on a change (e.g., decrease) of intensity of the optical signal between the inner surface and the outer surface of the skin flap during the optical signal transmission and/or an absorption of the optical signal by the skin flap during the optical signal transmission.

Method 800 may further include, at operation 806, determining whether a change as occurred in the one or more properties. In some implementations, determining whether a change in the one or more properties occurred may be determined by comparing the one or more properties to a baseline of the one or more properties. In instances where the one or more properties deviate from the baseline, a change in the one or more properties may be determined to have occurred. In some examples, a change in the one or more properties may be determined to have occurred when the one or more properties deviate from the baseline by a threshold amount.

In instances where a change in the one or more properties has not occurred (e.g., no, at operation 806), method 800 may include continuing to monitor the one or more properties such as by performing another optical signal transmission at operation 802. Alternatively, in instances where a change in the one or more properties has occurred (e.g., yes, at operation 806), method 800 may include, at operation 808, performing a mitigation operation associated with the skin flap. Such a mitigation operation may include providing a notification (e.g., by way of display 306) associated with the one or more properties and/or decreasing the strength of the one or more magnets, which may reduce the compression of the skin flap between the cochlear implant and the headpiece included in the cochlear implant system and prolong the health of the skin flap.

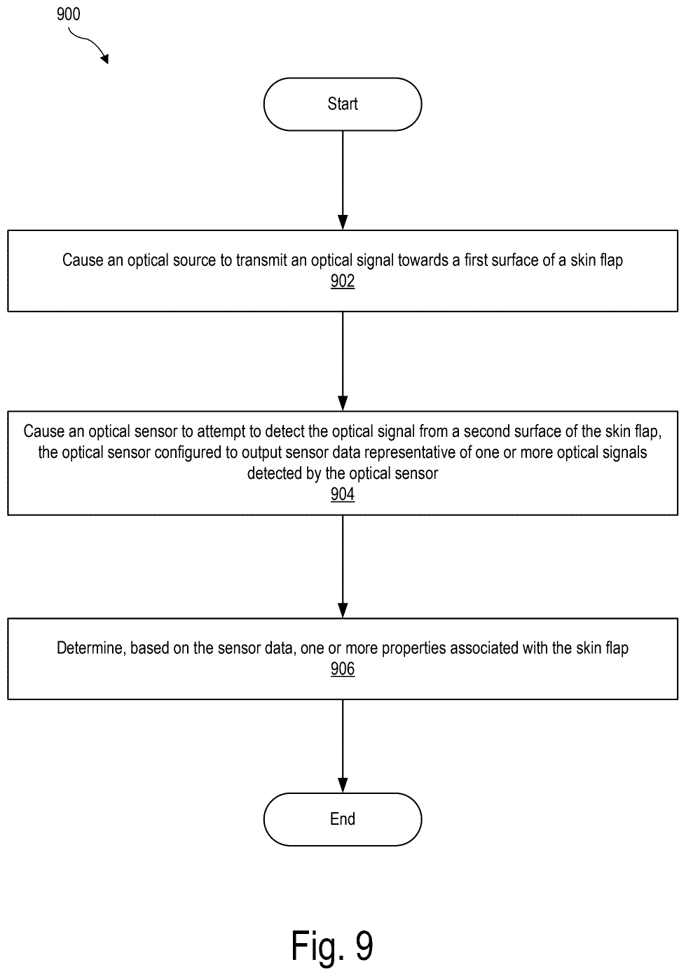

FIG. 9 depicts another illustrative method 900 for monitoring one or more properties associated with a skin flap (e.g., skin flap 212) based on an optical signal transmission of cochlear implant system 100. While FIG. 9 illustrates exemplary operations according to one embodiment, other embodiments may omit, add to, reorder, and/or modify any of the operations shown in FIG. 9. Moreover, each of the operations depicted in FIG. 9 may be performed in any of the ways described herein. One or more of the operations depicted in FIG. 9 may be performed by a processing unit (e.g., processing unit 108) of the cochlear implant system.

As shown, method 900 may include, at operation 902, causing an optical source (e.g., optical emitters 602) to transmit an optical signal (e.g., optical signal 702) towards a first surface of skin flap of a recipient. The optical source may be included in either an implantable device (e.g., cochlear implant 102) implanted within a recipient or an external device (e.g., headpiece 206) external to the recipient. In instances where the optical source is included within the implantable device, the optical source may transmit the optical signal towards the inner surface of the skin flap. Alternatively, in instances where the optical source is included within the external device, the optical source may transmit the optical signal towards the outer surface of the skin flap.

Method 900 may further include, at operation 904, causing an optical sensor (e.g., optical sensor 604) included in the other of the implantable device or the external device to attempt to detect the optical signal from a second surface of the skin flap of the recipient and through the skin flap. In instances where the optical sensor is included within the implantable device, the optical sensor may detect the optical signal at the inner surface of the skin flap. Alternatively, in instances where the optical sensor is included within the external device, the optical sensor may detect the optical signal at the outer surface of the skin flap. The optical sensor may further be configured to output sensor data representative of one or more optical signals detected by the optical sensor during a time period corresponding to the transmitting of the optical signal by the optical source.

Method 900 may further include, at operation 906, determining, based on the sensor data, one or more properties associated with the skin flap of the recipient. For example, the one or more properties may be determined based on the sensor data output by the one or more optical sensors. As an illustrative example, the sensor data may indicate a decrease in the intensity of the optical signal between the inner surface and the outer surface of the skin flap during the optical signal transmission such that the one or more properties may be determined based on the decrease in the intensity. Additionally or alternatively, the sensor data may indicate an amount of absorption of the optical signal between the inner surface and the outer surface of the skin flap during the optical signal transmission such that the one or more properties may be determined based on the amount of absorption.

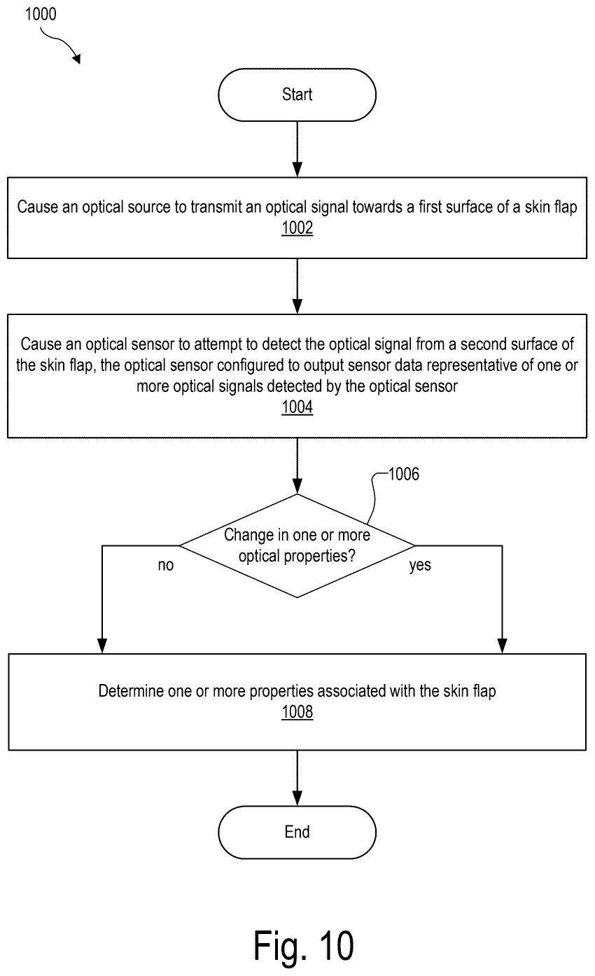

FIG. 10 depicts another illustrative method 1000 for monitoring one or more properties associated with a skin flap (e.g., skin flap 212) based on an optical signal transmission of cochlear implant system 100. While FIG. 10 illustrates exemplary operations according to one embodiment, other embodiments may omit, add to, reorder, and/or modify any of the operations shown in FIG. 10. Moreover, each of the operations depicted in FIG. 10 may be performed in any of the ways described herein. One or more of the operations depicted in FIG. 10 may be performed by a processing unit (e.g., processing unit 108) of the cochlear implant system.

As shown, method 1000 may include, at operation 1002, causing an optical source (e.g., optical emitters 602) to transmit an optical signal (e.g., optical signal 702) towards a first surface of skin flap of a recipient. The optical source may be included in either an implantable device (e.g., cochlear implant 102) implanted within a recipient or an external device (e.g., headpiece 206) external to the recipient. In instances where the optical source is included within the implantable device, the optical source may transmit the optical signal towards the inner surface of the skin flap. Alternatively, in instances where the optical source is included within the external device, the optical source may transmit the optical signal towards the outer surface of the skin flap. In some implementations, the optical signal may include one or more optical properties (e.g., intensity, wavelength, frequency, etc.) such that the optical signal may be transmitted from the optical source at one or more initial optical properties.

Method 1000 may further include, at operation 1004, causing an optical sensor (e.g., optical sensor 604) included in the other of the implantable device or the external device to attempt to detect the optical signal from a second surface of the skin flap of the recipient and through the skin flap. In instances where the optical sensor is included within the implantable device, the optical sensor may detect the optical signal at the inner surface of the skin flap. Alternatively, in instances where the optical sensor is included within the external device, the optical sensor may detect the optical signal at the outer surface of the skin flap. The optical sensor may further be configured to output sensor data representative of one or more optical signals detected by the optical sensor during a time period corresponding to the transmitting of the optical signal by the optical source.

Method 1000 may further include, at operation 1006, determining, based on the sensor data, whether a change occurred in the one or more optical properties (e.g., intensity, wavelength, frequency, etc.) of the optical signal during the optical signal transmission. For example, the cochlear implant system may determine one or more optical properties of the optical signal at the second surface of the skin flap based on the sensor data. The cochlear implant system may further compare such one or more optical properties to the initial one or more optical properties of the optical signal at the first surface of the skin flap based on the transmitted optical signal. If the detected one or more optical properties differ from the initial one or more properties, a change (e.g., an increase and/or a decrease) in the one or more optical properties may be determined. In some implementations, a change in the one or more optical properties may be determined to have occurred when the change exceeds a threshold amount.

In instances where a change in the one or more optical properties of the optical signal occurred during the optical signal transmission (e.g., yes, at operation 1006), method 1000 may further include, at operation 1008, determining one or more properties associated with the skin flap of the recipient. For example, the one or more properties associated with the skin flap may be determined based on the change of the one or more optical properties of the optical signal. As an illustrative example, the sensor data may indicate a decrease in the intensity of the optical signal between the inner surface and the outer surface of the skin flap during the optical signal transmission such that the one or more properties associated with the skin flap may be determined based on an amount of the decrease in the intensity. In some implementations, when the change in the one or more optical properties exceeds a threshold amount, the one or more properties associated with the skin may be determined to be associated with a healthy skin flap.

Alternatively, in instances where a change in the one or more optical properties of the optical signal has not occurred during the optical signal transmission (e.g., no, at operation 1006), method 1000 may also include, at operation 1008, determining one or more properties associated with the skin flap of the recipient. For example, the one or more properties associated with the skin flap may still be determined based on the one or more optical properties of the optical signal. However, a change in the one or more optical properties below a threshold amount may indicate that the one or more properties associated with the skin flap may be determined to be associated with damage to the skin flap (e.g., the skin flap may have a reduced thickness and/or blood circulation).

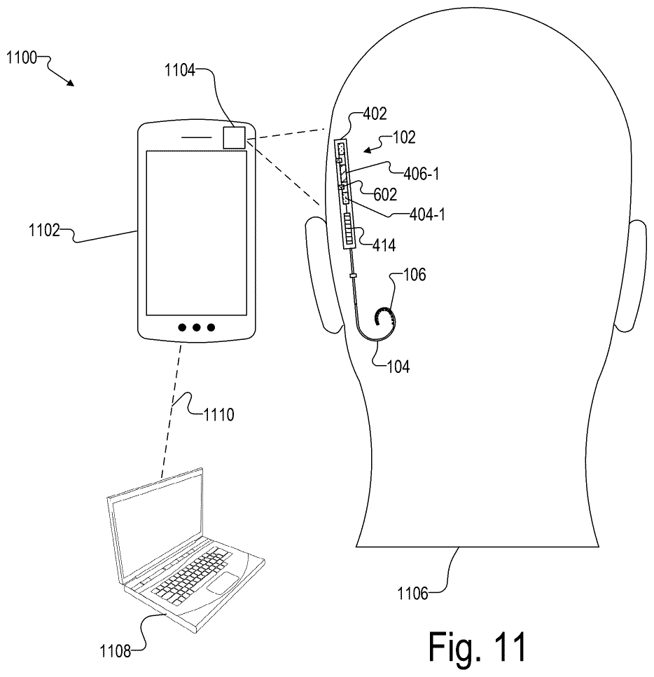

FIG. 11 shows another illustrative configuration 1100 of cochlear implant system 100 configured to monitor one or more properties associated with a skin flap based on an optical signal transmission. As depicted by the implementation in FIG. 11, the external device is implemented by a user end device 1102 instead of headpiece 206. Configuration 1100 may include additional or alternative components as may serve a particular implementation.

User end device 1102 is shown as a smartphone including an optical element 1104. However, user end device 1102 may be implemented by any suitable device separate from the cochlear implant system including an optical element 1104, such as a mobile device (e.g., a mobile phone, a laptop, a tablet computer, etc.). In the illustrated example, optical element 1104 of user end device 1102 is implemented by a camera such that optical element 1104 may image the implantation site of cochlear implant 102 during the optical signal transmission to detect the optical signal emitted from optical emitter 602. During use, optical emitter 602 of cochlear implant 102 may emit the optical signal through a skin flap at head 1106 of the recipient. User end device 1102 may be held at the implantation site of cochlear implant 102 with optical element 1104 positioned toward the skin flap at head 1106 of the recipient. Accordingly, the camera may image the implantation site to attempt to detect the optical signal from cochlear implant 102 and through the skin flap. For example, the camera may image light transmitted through the skin flap such as to detect an intensity and/or absorption of the light. User end device 1102 may generate sensor data representative of the detected optical signal.

In the illustrated example, user end device 1102 is communicatively coupled (e.g., wired and/or wirelessly) with a computing device 1108 such that user end device 1102 may output the sensor data to computing device 1108 by way of a back telemetry channel 1110. Computing device 1108 is shown as a laptop computer in FIG. 11 but could be implemented by any other suitable computing device such as described herein. Computing device 1108 may be configured to determine one or more properties associated with the skin flap based on the sensor data. In some other examples, user end device 1102 may implement computing device 1108 and determine the one or more properties based on the sensor data such that computing device 1108 may be omitted in some implementations.

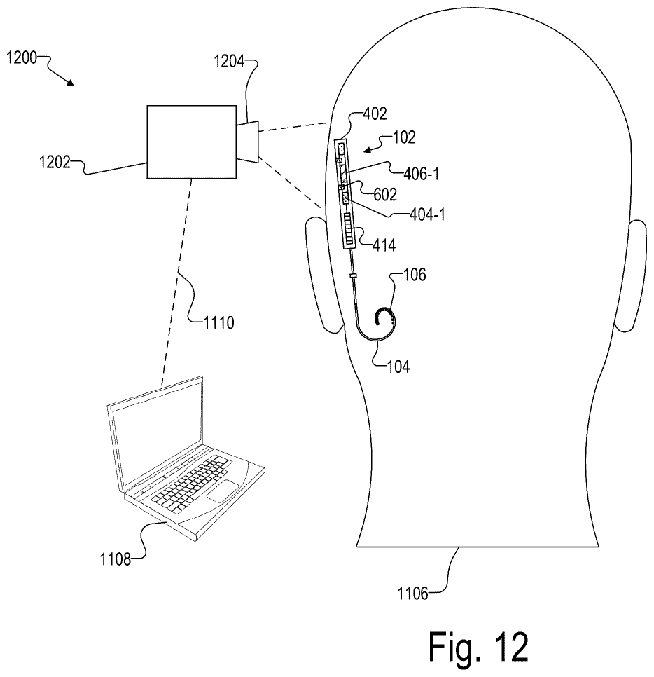

FIG. 12 shows another illustrative configuration 1200 of cochlear implant system 100 configured to monitor one or more properties associated with a skin flap based on an optical signal transmission. As depicted by the implementation in FIG. 12, the external device is implemented by an external headpiece 1202 configured to be used by a clinician such as for selectively performing the optical signal transmission instead of headpiece 206. Configuration 1200 may include additional or alternative components as may serve a particular implementation.

As shown, headpiece 1202 includes an optical element 1204, such as a camera, CCD array, photodetector, etc. During use, optical emitter 602 of cochlear implant 102 may emit the optical signal through a skin flap at head 1106 of the recipient. Headpiece 1202 may be held at the implantation site of cochlear implant 102 with optical element 1204 positioned toward the skin flap at head 1106 of the recipient. Accordingly, optical element 1204 may image the implantation site and/or attempt to detect the optical signal from cochlear implant 102 and through the skin flap. Headpiece 1202 may generate sensor data representative of the detected optical signal.

In the illustrated example, headpiece 1202 is communicatively coupled (e.g., wired and/or wirelessly) with computing device 1108 such that headpiece 1202 may output the sensor data to computing device 1108 by way of a back telemetry channel 1110. Computing device 1108 may be configured to determine one or more properties associated with the skin flap based on the sensor data. In some other examples, headpiece 1202 may implement computing device 1108 and determine the one or more properties based on the sensor data such that computing device 1108 may be omitted in some implementations.

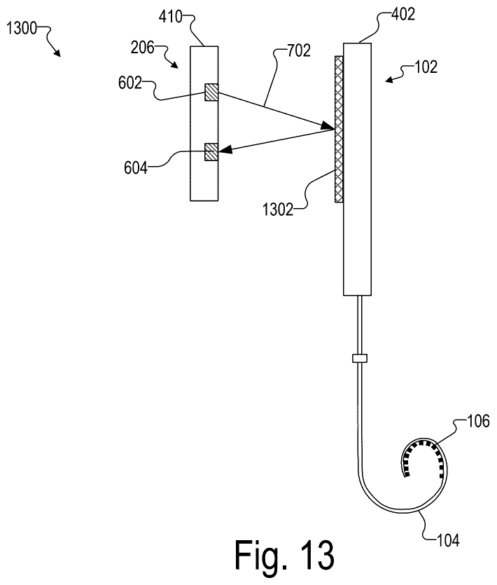

Still other suitable configurations for cochlear implant system 100 may be used. For example, FIG. 13 shows another illustrative configuration 1300 of cochlear implant system 100 configured to monitor one or more properties associated with a skin flap based on an optical signal transmission. As depicted by the implementation in FIG. 13, cochlear implant 102 includes a first optical element including a reflective material 1302 and an external device (e.g., headpiece 206) includes a second optical element including both an optical emitter 602 and an optical sensor 604. Configuration 1300 may include additional or alternative components as may serve a particular implementation.

Reflective material 1302 may be implemented by any suitable reflective material configured to reflect optical signal 702 such as light. In some implementations, the reflective material may be patterned. Reflective material 1302 may be positioned on a surface of cochlear implant 102 toward an inner surface of a skin flap, optical emitter 602, and optical sensor 604. During use, optical emitter 602 included in the external device may be configured to emit optical signal 702 through the skin flap and toward reflective material 1302 of cochlear implant 102. Reflective material 1302 may reflect at least a portion of optical signal 702 back through the skin flap and toward optical sensor 604 of the external device. Optical sensor 604 may attempt to detect the reflected optical signal 702 during the optical signal transmission such that the one or more properties associated with the skin flap may be determined based on the optical signal transmission.

Although the preceding description is described in the context of a cochlear implant system, it is understood that concepts such as those described herein may be applied in other contexts with other types of implantable stimulator systems.

In some examples, a non-transitory computer-readable medium storing computer-readable instructions may be provided in accordance with the principles described herein. The instructions, when executed by a processor of a computing device, may direct the processor and/or computing device to perform one or more operations, including one or more of the operations described herein. Such instructions may be stored and/or transmitted using any of a variety of known computer-readable media.

A non-transitory computer-readable medium as referred to herein may include any non-transitory storage medium that participates in providing data (e.g., instructions) that may be read and/or executed by a computing device (e.g., by a processor of a computing device). For example, a non-transitory computer-readable medium may include, but is not limited to, any combination of non-volatile storage media and/or volatile storage media. Exemplary non-volatile storage media include, but are not limited to, read-only memory, flash memory, a solid-state drive, a magnetic storage device (e.g., a hard disk, a floppy disk, magnetic tape, etc.), ferroelectric random-access memory (RAM), and an optical disc (e.g., a compact disc, a digital video disc, a Blu-ray disc, etc.). Exemplary volatile storage media include, but are not limited to, RAM (e.g., dynamic RAM).

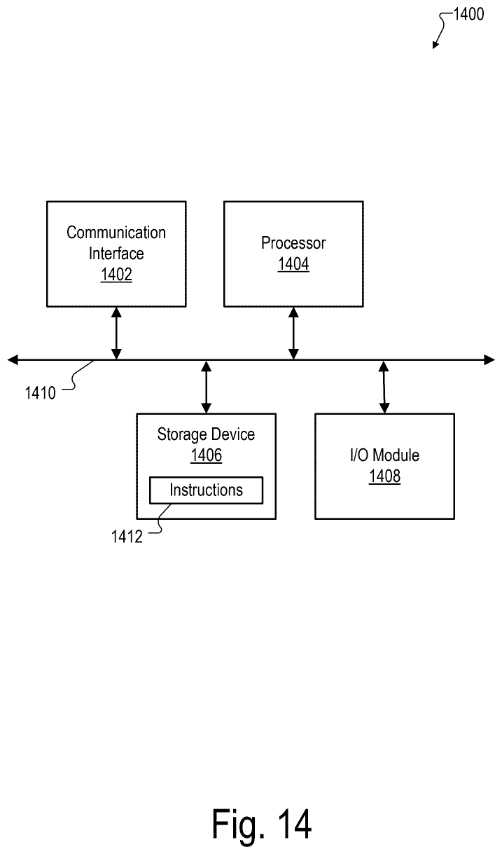

FIG. 14 illustrates an exemplary computing device 1400 that may be specifically configured to perform one or more of the processes described herein. As shown in FIG. 14, computing device 1400 may include a communication interface 1402, a processor 1404, a storage device 1406, and an input/output (I/O) module 1408 communicatively connected one to another via a communication infrastructure 1410. While an exemplary computing device 1400 is shown in FIG. 14, the components illustrated in FIG. 14 are not intended to be limiting. Additional or alternative components may be used in other embodiments. Components of computing device 1400 shown in FIG. 14 will now be described in additional detail.

Communication interface 1402 may be configured to communicate with one or more computing devices. Examples of communication interface 1402 include, without limitation, a wired network interface (such as a network interface card), a wireless network interface (such as a wireless network interface card), a modem, an audio/video connection, and any other suitable interface.

Processor 1404 generally represents any type or form of processing unit capable of processing data and/or interpreting, executing, and/or directing execution of one or more of the instructions, processes, and/or operations described herein. Processor 1404 may perform operations by executing computer-executable instructions 1412 (e.g., an application, software, code, and/or other executable data instance) stored in storage device 1406.

Storage device 1406 may include one or more data storage media, devices, or configurations and may employ any type, form, and combination of data storage media and/or device. For example, storage device 1406 may include, but is not limited to, any combination of the non-volatile media and/or volatile media described herein. Electronic data, including data described herein, may be temporarily and/or permanently stored in storage device 1406. For example, data representative of computer-executable instructions 1412 configured to direct processor 1404 to perform any of the operations described herein may be stored within storage device 1406. In some examples, data may be arranged in one or more databases residing within storage device 1406.

I/O module 1408 may include one or more I/O modules configured to receive user input and provide user output. One or more I/O modules may be used to receive input for a virtual experience. I/O module 1408 may include any hardware, firmware, software, or combination thereof supportive of input and output capabilities. For example, I/O module 1408 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 1408 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 1408 is configured to provide graphical data to a display for presentation to a user. The graphical data may be representative of one or more graphical user interfaces and/or any other graphical content as may serve a particular implementation. In certain embodiments, I/O module 1408 may be configured to provide a trigger signal associated with any of the operations described herein. For example, the trigger signal provided by I/O module 1408 may be used to facilitate detecting evoked responses during bilateral stimulation.

In some examples, any of the systems, computing devices, and/or other components described herein may be implemented by computing device 1400. For example, memory 112 may be implemented by storage device 1406, and processor 114 may be implemented by processor 1404.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

What is claimed is:

1. A cochlear implant system comprising:
a cochlear implant comprising a first optical element configured to be implanted within a recipient and positioned toward an inner surface of a skin flap of the recipient; and
a processing unit communicatively coupled with the cochlear implant and configured to:
cause an optical signal transmission between the first optical element and a second optical element included in an external device and configured to be positioned toward outer surface of the skin flap relative to the first optical element, the optical signal transmission including a transmission of an optical signal through the skin flap between the inner surface and the outer surface of the skin flap; and
determine, based on the optical signal transmission, one or more properties associated with the skin flap of the recipient.

2. The cochlear implant system of claim 1, wherein one of the first optical element or the second optical element includes an optical emitter configured to transmit the optical signal.

3. The cochlear implant system of claim 2, wherein the transmitting the optical signal includes modulating the optical signal.

4. The cochlear implant system of claim 2, wherein the optical emitter includes one or more light-emitting diodes (LEDs).

5. The cochlear implant system of claim 4, wherein the one or more LEDs are multispectral.

6. The cochlear implant system of claim 1, wherein the first optical element is positioned at one or both of an antenna or a magnet included in the cochlear implant.

7. The cochlear implant system of claim 1, wherein one of the first optical element or the second optical element includes an optical sensor configured to attempt to detect the optical signal during the optical signal transmission.

8. The cochlear implant system of claim 7, wherein the optical sensor comprises one or more of a camera, a charged-coupled device (CCD), or a photodetector.

9. The cochlear implant system of claim 1, wherein the external device includes a headpiece of the cochlear implant system and configured to be worn by the recipient.

10. The cochlear implant system of claim 9, wherein the second optical element is positioned at one or both of an antenna or a magnet included in the headpiece.

11. The cochlear implant system of claim 9, wherein the second optical element is aligned with the first optical element when the headpiece is magnetically coupled to the cochlear implant in any rotational position of the headpiece relative to the cochlear implant.

12. The cochlear implant system of claim 1, wherein the external device is separate from the cochlear implant system.

13. The cochlear implant system of claim 1, wherein the processing unit is included in a sound processor of the cochlear implant system.

14. The cochlear implant system of claim 1, wherein the one or more properties associated with the skin flap include one or more of a thickness, a blood oxygenation, a pulse rate, a blood vessel size, a blood flow, a blood perfusion, or a blood pressure.

15. The cochlear implant system of claim 1, wherein the determining the one or more properties is based on one or more of an absorption of the optical signal or a change of intensity of the optical signal between the inner surface and the outer surface of the skin flap during the optical signal transmission.

16. The cochlear implant system of claim 1, wherein the determining the one or more properties includes determining a change in the one or more properties relative to a baseline associated with the one or more properties.

17. The cochlear implant system of claim 16, further comprising providing a notification associated with the change in the one or more properties.

18. The cochlear implant system of claim 16, further comprising decreasing a strength of one or more magnets included in the cochlear implant system.

19. A cochlear implant system comprising:
a cochlear implant comprising a first optical element configured to be implanted within a recipient and positioned toward an inner surface of a skin flap of the recipient;
a headpiece comprising a second optical configured to be positioned external of the recipient toward an outer surface of the skin flap relative to the first optical element; and
a processing unit communicatively coupled with the cochlear implant and the headpiece, the processing unit configured to:
cause an optical signal transmission between the first optical element and the second optical element, the optical signal transmission including a transmission of an optical signal through the skin flap between the inner surface and the outer surface of the skin flap; and
determine, based on the optical signal transmission, one or more properties associated with the skin flap of the recipient.

20. A system comprising:
a memory storing instructions; and
a processor configured to execute the instructions to perform a process comprising:
causing an optical source to transmit an optical signal towards a first surface of skin flap of a recipient, the optical source included in either an implantable device implanted within a recipient or an external device external to the recipient;
causing an optical sensor included in the other of the implantable device or the external device to attempt to detect the optical signal from a second surface of the skin flap of the recipient and through the skin flap, the optical sensor configured to output sensor data representative of one or more optical signals detected by the optical sensor during a time period corresponding to the transmitting of the optical signal by the optical source; and
determining, based on the sensor data, one or more properties associated with the skin flap of the recipient.
